(19) United States
(12) Patent Application Publication
(10) Pub. No.: US 2011/0125516 A1
(43) Pub. Date: May 26, 2011

(54) SYSTEM AND METHOD FOR PURCHASE AND DISTRIBUTION MANAGING OF HOSPITAL ARTICLES

(75) Inventor: Jae Hoon Choi, Gyeonggi-do (KR)

(73) Assignee: EZMEDICOM CO., LTD., Seoul (KR)

(21) Appl. No.: 12/674,302

(22) PCT Filed: Feb. 23, 2009
(86) PCT No.: PCT/KR09/00845

§ 371 Date: Feb. 19, 2010

(30) Foreign Application Priority Data
Feb. 25, 2008 (KR) 10-2008-0016850

(51) Int. Cl. G06Q 10/00; G06Q 30/00; G06Q 50/00

(52) U.S. Cl. 705/2; 705/26.41

(57) ABSTRACT

There are provided a purchase and distribution management system and method for purchasing articles used in a hospital by proxy and managing the distribution of the articles, the purchase and distribution management system including: an interface part connecting an exclusive management device of the hospital, the device computerizing the articles in the hospital, to the purchase and distribution management system to be compatible with each other in such a way that a user is able to use the purchase and distribution management system by using only the exclusive management device without downloading a new program; a purchase application reception part receiving a purchase application for hospital articles from the user; a contract check part retrieving whether there exists a contract between the user and a provider for the articles; a contract generation part generating the contract between the user and the provider when there is no contract for the hospital articles for which purchase application is received as a result of the retrieving of the contract check part; a purchase order management part generating and sending a purchase order for the articles to the provider when there is the contract for the articles for which purchase application is received as the result of the retrieving of the contract check part or the contract is generated by the contract generation part; a distribution management part checking an amount of each article in stock in the hospital, generating a purchase application according to a result of the checking, and managing a delivery process of the article in the hospital; and a consignment stock management part allowing a certain amount of warehoused articles for consignment stock to be consigned to the provider to be stored and changing an amount of interim warehoused articles consigned to the provider according to a use of the user.

### TECHNICAL FIELD

The present invention relates to a system and method for purchasing hospital articles and managing distribution of the hospital articles, and more particularly, to a system and method for purchasing articles used in a hospital by proxy and managing distribution of the articles in the hospital.

### BACKGROUND ART

Recently, as scientific technologies develop, various kinds of articles for hospital are generated. According to this trend, the whole amount of hospital article transaction also rapidly increases.

Such transaction for hospital articles is generally performed on an offline contract between a purchaser and a seller. However, lately, as Internet technologies are quickly spread, websites for purchasing and selling hospital articles, capable of realizing a contract between a purchaser and a seller online open based on Internet environments.

For example, Korean Patent Application No. 2000-0036933 discloses Method of providing hospital article purchase system in computer network system and a recording medium for executing the method in which there is disclosed a conventional hospital article purchasing and selling website in detail.

However, conventional hospital article purchasing and selling websites execute no more than relay a transaction for hospital articles between a plurality of general purchasers and sellers. Generally, there is no merit except for relaying the transaction for hospital articles between purchasers and sellers on line. Also, according to conventional technologies, there is inconvenience that it is possible to use a purchase system by loading a program provided in addition to an exclusive management device of each hospital, that is, a management program. Also, it is required to additionally manage distribution of hospital articles purchased via a conventional website in a hospital after inputting hospital articles in a hospital computation system.

### DISCLOSURE OF INVENTION

### Technical Problem

The present invention provides a purchase and distribution management system and method allowing a user to purchase hospital articles and manage distribution of the hospital articles by using an exclusive management device of each hospital.

The present invention also provides a purchase and distribution management system and method capable of purchasing hospital articles by proxy simultaneously with managing distribution of the purchased hospital articles in the each hospital.

The present invention also provides a purchase and distribution management system and method capable of providing a uniform amount of articles in stock by generating interim warehousing simultaneously with providing consignment stock management to reduce a storage space.

### Technical Solution

According to an aspect of the present invention, there is provided a purchase and distribution management system for purchasing articles used in a hospital by proxy and managing the distribution of the articles, the system including: an interface part connecting an exclusive management device of the hospital, the device computerizing the articles in the hospital, to the purchase and distribution management system to be compatible with each other in such a way that a user is able to use the purchase and distribution management system by using only the exclusive management device without downloading a new program; a purchase application reception part receiving a purchase application for hospital articles from the user; a contract check part retrieving whether there exists a contract between the user, and a provider for the articles; a contract generation part generating the contract between the user and the provider when there is no contract for the hospital articles for which purchase application is received as a result of the retrieving of the contract check part; a purchase order management part generating and sending a purchase order for the articles to the provider when there is the contract for the articles for which purchase application is received as the result of the retrieving of the contract check part or the contract is generated by the contract generation part; a distribution management part checking an amount of each article in stock in the hospital, generating a purchase application according to a result of the checking, and managing a delivery process of the article in the hospital; and a consignment stock management part allowing a certain amount of warehoused articles for consignment stock to be consigned to the provider to be stored and changing an amount of interim warehoused articles consigned to the provider according to a use of the user.

The system may further include a warehousing check part checking one of warehousing and interim warehousing of articles ordered by the purchase order management part.

The contract generation part may include: a contract type selection part selecting one of a private contract and a tender contract; a private contract part requesting and receiving an estimate and making a contract with the provider when the contract type selection part selects the private contract; a tender contract part receiving tenders from a plurality of providers and making a contract when the contract type selection part selects the tender contract; and a contract approval part receiving an approval of the user with respect to a contract made by one of the private contract part and the tender contract part.

The tender contract part may include: a tender notice generation and providing part generating a notice of tender based on the purchase application of the user and providing the notice of tender to the plurality of providers; a tender application receiving part receiving tender applications from the plurality of providers; and a tender examination part examining the tender applications received by the tender application receiving part, accepting one of the tender applications and making a contract with a provider applying the accepted tender application.

The tender examination part may group a plurality of items, may examine the tender applications for each group, accept one of the tender applications, and make a contract with the provider applying the accepted tender application.

The purchase order management part may include: a purchase order generation part generating a purchase order on the articles when there is the contract for the articles for which purchase application is received as the result of the retrieving of the contract check part or the contract is generated by the contract generation part; an electronic payment part allowing the user to excuse an electronic payment with respect to an amount of money written in the purchase order; and a purchase order sending part sending the purchase order generated by the purchase order generation part to the provider.

The distribution management part may include one or more of: a prescription article management part, when a prescription on a prescription article is inputted, adding the prescription up, generating a delivery list for each department, and executing a daily delivery; a prepayment article management part, when a prescription on a particular prepayment article is inputted for each patient, generating a purchase application according to the prescription, sending the prescription to the purchase application reception part, and transmitting a prepayment request signal to the purchase order management part; a quantitative supplement article management part checking a stock reference amount, a use amount, an amount of articles in stock, and a delivery amount for each department with respect to nonprescription articles and executing a delivery of the nonprescription articles to uniformly maintain the stock reference amount of the each department; and a consumptive article management part receiving an article application with respect to consumptive articles used in hospital from the each department, executing a delivery of a corresponding article, and forming data of the delivery amount.

The consignment stock management part may include: an object article check part checking which article is an object of consignment stock management; a delivery investigation part investigating a delivery amount of the checked article to the each department; an interim warehousing generation part sending the purchase application to the purchase application reception part to control a warehousing amount and an interim warehousing amount within a predetermined range, based on information from the delivery investigation part; and a consignment stock control part checking the ware housing amount and the interim warehousing amount and providing control information to control a ratio between the warehousing amount and the interim warehousing amount within a predetermined range.

The system may further include a standardization code mapping part receiving internal codes of the user and the provider with respect to hospital articles, matching the internal codes with standardization codes, and providing information on the matched standardization codes to the user and the provider.

The standardization code mapping part may include: a code reception part receiving the internal codes of the user and the provider with respect to the hospital articles; a standardization code database storing standardization codes corresponding to hospital articles; a code matching part matching the internal code with the standardization code; and a code relation database storing information on a relation between the matched internal code and standardization code.

The system may further include a barcode management part providing a barcode including information on a hospital article to allow the amount of the hospital articles in stock to be efficiently managed.

The barcode management part may include: a barcode generation part generating the barcode including information on the hospital article; and a barcode output part outputting and providing the generated barcode.

The information on the hospital article may include one or more of a validity period, a contract period, an insurance maximum price, a load position, a price, distribution, and a standardization code of the hospital article.

According to another aspect of the present invention, there is provided a purchase and distribution management method for purchasing articles used in a hospital by proxy and managing the distribution of the articles by using the purchase and distribution management system, the method including: connecting an exclusive management device of the hospital, the device computerizing the articles in the hospital, to the purchase and distribution management system to be compatible with each other in such a way that a user is able to use the purchase and distribution management system by using only the exclusive management device without downloading a new program; receiving a purchase application for hospital articles from the user; retrieving whether there exists a contract between the user and a provider for the articles; generating the contract between the user and the provider when there is no contract for the hospital articles for which purchase application is received as a result of the retrieving of the contract check part; generating and sending a purchase order for the articles to the provider when there is the contract for the articles for which purchase application is received as the result of the retrieving of the contract check part or the contract is generated by the contract generation part; checking an amount of each article in stock in the hospital, generating a purchase application according to a result of the checking, and managing a delivery process of the article in the hospital; and allowing a certain amount of warehoused articles for consignment stock to be consigned to the provider to be stored and changing an amount of interim warehoused articles consigned to the provider according to a use of the user.

The method may further include checking one of warehousing and interim warehousing of articles ordered in the generating and sending a purchase order.

The generating the contract may include: selecting one of a private contract and a tender contract; requesting and receiving an estimate and making a private contract with the provider when the private contract is selected; receiving tenders from a plurality of providers and making a tender contract when the tender contract is selected; and receiving an approval of the user with respect to the contract made in the above.

The receiving tenders from a plurality of providers and making a tender contract may include: generating a notice of tender based on the purchase application of the user and providing the notice of tender to the plurality of providers; receiving tender applications from the plurality of providers; and examining the received tender applications, accepting one of the tender applications and making a contract with a provider applying the accepted tender application.

In the examining the received tender applications, a plurality of items may be grouped, the tender applications may be examined for each group, one of the tender applications may be accepted, and a contract with the provider applying the accepted tender application may be made.

The generating and sending a purchase order may include: generating a purchase order on the articles when there is the contract for the articles for which purchase application is received as the result of the retrieving of the contract or the contract is generated in the above; allowing the user to excuse an electronic payment with respect to an amount of money written in the purchase order; and sending the generated purchase order to the provider.

The managing a delivery process may include one or more of: when a prescription on a prescription article is inputted, adding the prescription up, generating a delivery list for each department, and executing a daily delivery; when a prescription on a particular prepayment article is inputted for each patient, generating a purchase application according to the prescription, sending the prescription to the purchase application reception part, and transmitting a prepayment request signal to the purchase order management part; checking a stock reference amount, a use amount, an amount of articles in stock, and a delivery amount for each department with respect to nonprescription articles and executing a delivery of the nonprescription articles to uniformly maintain the stock reference amount of the each department; and receiving an article application with respect to consumptive articles used in hospital from the each department, executing a delivery of a corresponding article, and forming data of the delivery amount.

The allowing a certain amount of warehoused articles for consignment stock to be consigned to the provider may include: checking which article is an object of consignment stock management; investigating a delivery amount of the checked article to the each department; sending the purchase application to the purchase application reception part to control a warehousing amount and an interim warehousing amount within a predetermined range, based on information from the investigating a delivery amount; and checking the ware housing amount and the interim warehousing amount and providing control information to control a ratio between the warehousing amount and the interim warehousing amount within a predetermined range.

The method may further include receiving internal codes of the user and the provider with respect to hospital articles, matching the internal codes with standardization codes, and providing information on the matched standardization codes to the user and the provider.

The receiving internal codes of the user and the provider with respect to hospital articles, matching the internal codes with standardization codes, and providing information on the matched standardization codes to the user and the provider may include: receiving the internal codes of the user and the provider with respect to the hospital articles; storing standardization codes corresponding to hospital articles in a standardization code database; matching the internal code with the standardization code; and storing information on a relation between the matched internal code and standardization code in a code relation database.

The method may further include providing a barcode including information on a hospital article to allow the amount of the hospital articles in stock to be efficiently managed. The providing a barcode including information on a hospital article to allow the amount of the hospital articles in stock to be efficiently managed may include: generating the barcode including information on the hospital article; and outputting and providing the generated barcode. The information on the hospital article may include one or more of a validity period, a contract period, an insurance maximum price, a load position, a price, distribution, and a standardization code of the hospital article.

### ADVANTAGEOUS EFFECTS

As described above, in the purchase and distribution management system and method according to an embodiment of the present invention, there are many excellent advantages as follows.

First, according to the purchase and distribution management system and method according to an embodiment of the present invention, a user may purchase hospital articles and manage distribution of the hospital articles by using an exclusive management device in each hospital without loading an additional program.

Second, according to the purchase and distribution management system and method according to an embodiment of the present invention, not only the hospital articles may be purchased by proxy but also the distribution of the hospital articles in the hospital may be managed.

Third, according to the purchase and distribution management system and method according to an embodiment of the present invention, interim warehousing is generated, thereby providing a certain amount of articles in stock simultaneously with reducing a storage space.

Fourth, according to the purchase and distribution management system and method according to an embodiment of the present invention, the user may easily receive data on the present status of purchasing hospital articles and delivering the hospital articles in a hospital, thereby helping establishing a plan for future article administration.

Fifth, according to the purchase and distribution management system and method according to an embodiment of the present invention, internal codes of a user and a provider with respect to hospital articles are received and matched with standardization codes and the matched standardization codes are provided to the user and the provider, thereby efficiently managing distribution of the hospital articles.

Sixth, according to the purchase and distribution management system and method according to an embodiment of the present invention, a barcode including information on hospital articles is provided to efficiently manage the hospital articles in stock.

### BRIEF DESCRIPTION OF DRAWINGS

FIG. 1 is a schematic diagram illustrating a whole configuration including a purchase and distribution management system according to an embodiment of the present invention;

FIG. 2 is a block diagram illustrating a configuration of the purchase and distribution management system;

FIG. 3 is a block diagram illustrating a configuration of a tender contract part according to an embodiment of the present invention;

FIG. 4 is a block diagram illustrating a configuration of a consignment stock management part according to an embodiment of the present invention;

FIG. 5 is a flowchart illustrating a purchase and distribution management method according to an embodiment of the present invention;

FIG. 6 is a flowchart illustrating a step of generating a contract, according to an embodiment of the present invention; and

FIG. 7 is a flowchart illustrating a step of managing a consignment stock, according to an embodiment of the present invention.

### BEST MODE FOR CARRYING OUT THE INVENTION

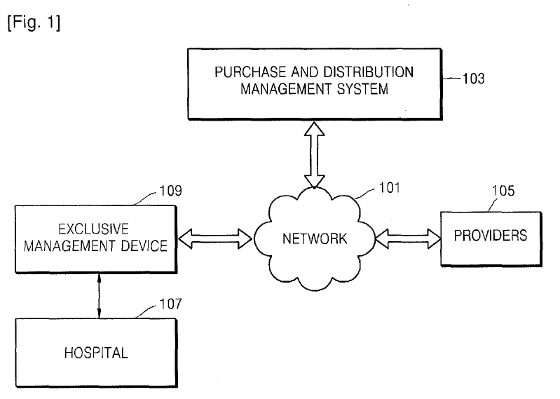

Hereinafter, exemplary embodiments of the present invention will be described in detail with reference to the attached drawings. FIG. 1 is a schematic diagram illustrating a whole configuration including a purchase and distribution management system according to an embodiment of the present invention 103.

Referring to FIG. 1, the whole configuration includes a network 101, the purchase and distribution management system 103, a plurality of providers 105, a user, that is, a hospital 107, and a management device 109 of the hospital 107.

The plurality of providers 105 is connected to the purchase and distribution management system 103 via the network 101, and the hospital 107 is connected to the network 101 by using the existing management device 109 instead of using another program.

The purchase and distribution management system 103 is connected to the plurality of providers 105, the hospital 107, and the management device 109, via the network 101.

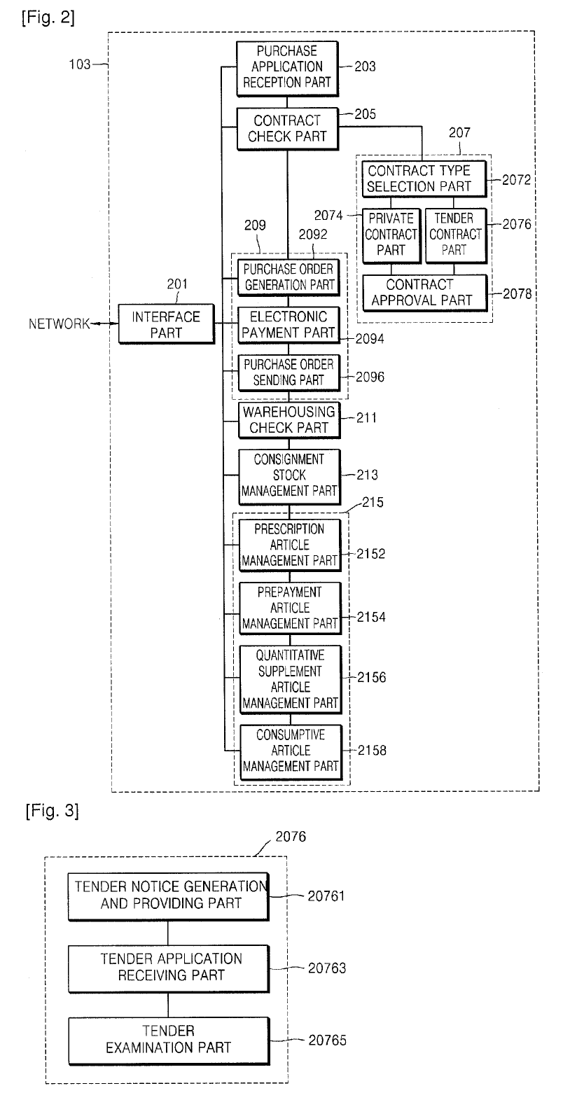

FIG. 2 is a block diagram illustrating a configuration of the purchase and distribution management system 103.

Referring to FIG. 2, the purchase and distribution management system 103 purchases articles used in hospital by proxy and manages distribution of the articles in hospital. The purchase and distribution management system 103 includes an interface part 201, a purchase application reception part 203, a contract check part 205, a contract generation part 207, a purchase order management part 209, a warehousing check part 211, a consignment stock management part 213, and a distribution management part 215. The interface part 201 connects the management device 109, which is an exclusive device in each hospital 107 and computerizes articles in hospital, to the purchase distribution management system 103 to be compatible with each other in such a way that the user, that is, the hospital 107 may use the purchase and distribution management system 103 by using only the management device 109.

The interface part 201 may be, for example, an interface program. Because of the interface part 201, the user may avoid inconvenience of downloading a new program to use the purchase and distribution management system 103.

The purchase and distribution management system 103 may further include a standardization code mapping part (not shown). The standardization code mapping part receives internal codes of a user and a provider with respect to hospital articles matches the internal codes with standardization codes, and provides information on the matched standardization codes to the user and the provider. Matching the internal codes with the standardization codes may make a connection between the interface part 201 and the management device 109 easy.

The standardization code mapping part includes a code reception part (not shown) receiving the internal codes of the user and the provider with respect to the hospital articles, a standardization code database (not shown) storing standardization codes corresponding to hospital articles, a code matching part (not shown) matching the internal code with the standardization code, and a code relation database (not shown) storing information on a relation between the matched internal code and standardization code.

The purchase application reception part 203 receives a purchase application for hospital articles from the user. For example, when the user applies a purchase by using the management device 109, the interface part 201 converts the purchase application into an application signal and transmits the application signal to the purchase application reception part 203. The contract check part 205 retrieves whether there is a contract between the user and a provider for the articles.

The contract generation part 207 generates the contract between the user and the provider when there is no contract for the hospital articles for which purchase application is received as a result of the retrieving of the contract check part 205. The contract generation part 207 includes a contract type selection part 2072, a private contract part 2074, a tender contract part 2076, and a contract approval part 2078.

The contract type selection part 2072 selects one of a private contract and a tender contract according to a certain basis. The certain basis may include, but not limited thereto, a total amount of a contract and a kind of an article.

The private contract part 2074 requests and receives an estimate and making a contract with the provider when the contract type selection part 2072 selects the private contract. The tender contract part 2076 receives tenders from a plurality of providers and makes a contract when the contract type selection part 2072 selects the tender contract. The contract approval part 2078 receives an approval of the user with respect to a contract made by one of the private contract part 2074 and the tender contract part 2076.

The purchase order management part 209 generates and sends a purchase order for the articles to the provider when there is the contract for the articles for which purchase application is received as the result of the retrieving of the contract check part 205 or the contract is generated by the contract generation part 207. The purchase order management part 209 includes a purchase order generation part 2092, an electronic payment part 2094, and a purchase order sending part 2096.

The purchase order generation part 2092 generates a purchase order on the articles when there is the contract for the articles for which purchase application is received as the result of the retrieving of the contract check part 205 or the contract is generated by the contract generation part 207. The electronic payment part 2094 allows the user to excuse an electronic payment with respect to an amount of money written in the purchase order. The purchase order sending part 2096 sends the purchase order generated by the purchase order generation part 2092 to the provider.

The provider receiving the purchase order warehouses corresponding articles into the hospital within a designated period.

The warehousing check part 211 checks one of warehousing and interim warehousing of the articles ordered by the purchase order management part 209.

The consignment stock management part 213 allows a certain amount of warehoused articles for consignment stock to be consigned to the provider to be stored and changes an amount of interim warehoused articles consigned to the provider according to a use of the user. The distribution management part 215 checks an amount of each article in stock in the hospital, generates a purchase application according to a result of the checking, and manages a delivery process of the article in the hospital.

The distribution management part 215 may employ various management processes according to the kind of an article. The distribution management part 215 includes a prescription article management part 2152 for prescription medical supplies, a prepayment article management part 2154 for medical supplies requiring prepayment, a quantitative supplement article management part 2156 for providing a certain amount of nonprescription articles in a department, and a consumptive article management part 2158 for other administration supplies required in hospital.

The prescription article management part 2152, when a prescription on a prescription article is inputted, adds the prescription up, generates a delivery list for each department, and executes a daily delivery. The prepayment article management part 2154, when a prescription on a particular prepayment article is inputted for each patient, generates a purchase application according to the prescription, sends the prescription to the purchase application reception part 203, and transmits a prepayment request signal to the purchase order management part 209. The quantitative supplement article management part 2156 checks a stock reference amount, a use amount, an amount of articles in stock, and a delivery amount for each department with respect to nonprescription articles and executes a delivery of the nonprescription articles to uniformly maintain the stock reference amount of the each department.

The consumptive article management part 2158 receives an article application with respect to consumptive articles used in hospital from the each department, executes a delivery of a corresponding article, and forms data of the delivery amount.

FIG. 3 is a block diagram illustrating a configuration of the tender contract part 2076.

Referring to FIG. 3, the tender contract part 2076 includes a tender notice generation and providing part 20761, a tender application receiving part 20763, and a tender examination part 20765. The tender notice generation and providing part 20761 generates a notice of tender based on the purchase application of the user and provides the notice of tender to the plurality of providers. The tender application receiving part 20763 receives tender applications from the plurality of providers.

The tender examination part 20765 examines the tender applications received by the tender application receiving part 20763, accepts one of the tender applications and making a contract with a provider applying the accepted tender application.

The tender examination part 20765 may group a plurality of items, examine the tender applications for each group, accept one of the tender applications, and make a contract with the provider applying the accepted tender application. The tender examination part 20765 may examine tender applications with respect to one of whole articles or each article, accept one of the tender applications, and make a contract with the provider applying the accepted tender application.

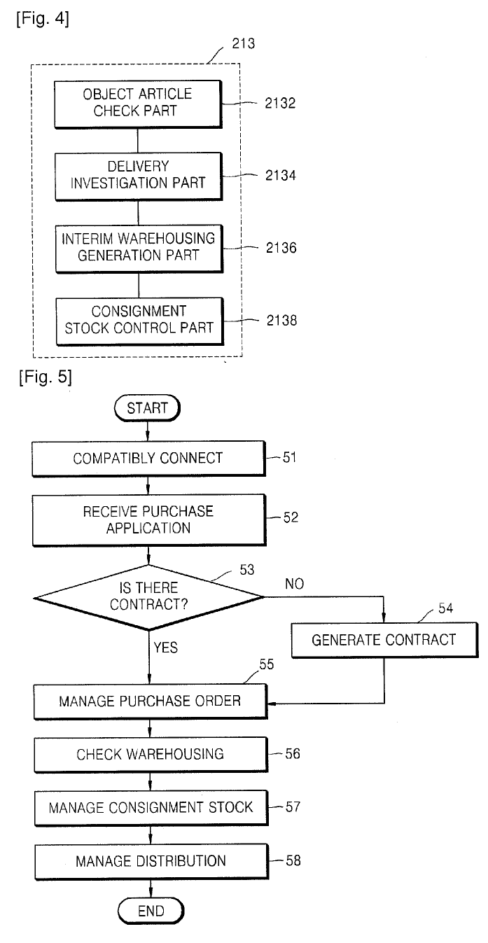

FIG. 4 is a block diagram illustrating a configuration of the consignment stock management part 213.

Referring to FIG. 4, the consignment stock management part 213 includes an object article check part 2132, a delivery investigation part 2134, an interim warehousing generation part 2136, and a consignment stock control part 2138. The object article check part 2132 checks which article is an object of consignment stock management. When an article is determined to be the object, a consignment stock process is performed. The delivery investigation part 2134 refers a delivery amount of the checked article to the each department. The interim warehousing generation part 2136 sends the purchase application to the purchase application reception part 203 to control a warehousing amount and an interim warehousing amount within a predetermined range, based on information from the delivery investigation part 2134. The consignment stock control part 2138 checks the ware housing amount and the interim warehousing amount and provides control information to control a ratio between the warehousing amount and the interim warehousing amount within a predetermined range.

FIG. 5 is a flowchart illustrating a purchase and distribution management method according to an embodiment of the present invention.

Referring to FIG. 5, the purchase and distribution management method includes connecting the exclusive management device 109 of each hospital, the device 109 computerizing the articles in the hospital, to the purchase and distribution management system 103 to be compatible with each other in such a way that a user may use the purchase and distribution management system 103 by using only the exclusive management device 109 (51), receiving a purchase application for hospital articles from the user (52), retrieving whether there exists a contract between the user and a provider for the articles (53), generating a contract between the user and the provider (54) when there is no contract for the hospital articles for which purchase application is received as a result of the retrieving.

On the other hand, when there is a contract for the hospital articles for which purchase application is received as the result of the retrieving or the contract is generated in S54, a purchase order for the articles is generated and sent to the provider (55). In S55, a purchase order on the articles may be generated when there is the contract for the articles for which purchase application is received as the result of the retrieving of the contract or the contract is generated in the above, the user may excuse an electronic payment with respect to an amount of money written in the purchase order, and the generated purchase order may be sent to the provider.

After this, in S56, warehousing or interim warehousing of the article ordered in S55 is checked. Also, in S57, a certain amount of warehoused articles for consignment stock is consigned to the provider to be stored and an amount of interim warehoused articles consigned to the provider is changed according to a use of the user. Also, in S58, an amount of each article in stock in the hospital is checked, a purchase application is generated according to a result of the checking, and a delivery process of the article in the hospital is managed.

The managing a delivery process (58) may include one or more of: when a prescription on a prescription article is inputted, adding the prescription up, generating a delivery list for each department, and executing a daily delivery; when a prescription on a particular prepayment article is inputted for each patient, generating a purchase application according to the prescription, sending the prescription to the purchase application reception part 203, and transmitting a prepayment request signal to the purchase order management part 209; checking a stock reference amount, a use amount, an amount of articles in stock, and a delivery amount for each department with respect to nonprescription articles and executing a delivery of the nonprescription articles to uniformly maintain the stock reference amount of the each department; and receiving an article application with respect to consumptive articles used in hospital from the each department, executing a delivery of a corresponding article, and forming data of the delivery amount.

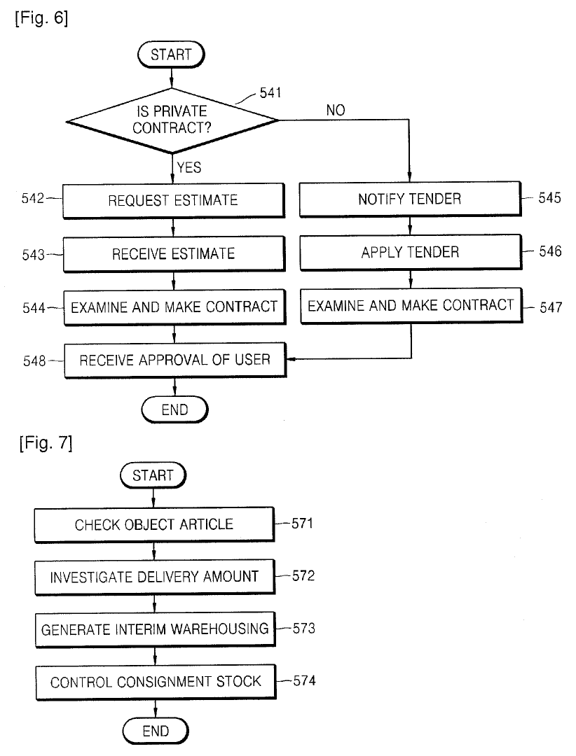

FIG. 6 is a flowchart illustrating sub-operations of the generating a contract (54).

Referring to FIG. 6, in the generating a contract (54), one of a private contract and a tender contract is selected (541). When the private contract is selected, an estimate is requested a provider (542) and received from the provider (543) and a private contract with the provider is made (544). On the other hand, when the tender contract is selected, a notice of tender is generated based on the purchase application of the user and provided to a plurality of providers (545), tender applications are received from the plurality of providers (S546), and the received tender applications are examined, one of the tender applications is accepted and a contract with a provider applying the accepted tender application is made (547).

In the examining the received tender applications (547), a plurality of items may be grouped, the tender applications may be examined for each group, one of the tender applications may be accepted, and a contract with the provider applying the accepted tender application may be made. After this, an approval of the user with respect to the contract made in 547 is received (548).

FIG. 7 is a flowchart illustrating sub-operations of the consignment stock management (57).

Referring to FIG. 7, the consignment stock management 57 includes: checking which article is an object of consignment stock management (571); investigating a delivery amount of the checked article for the each department (572); sending the purchase application to the purchase application reception part 203 to control a warehousing amount and an interim warehousing amount within a predetermined range, based on information from the investigating a delivery amount (573); and checking the ware housing amount and the interim warehousing amount and providing control information to control a ratio between the warehousing amount and the interim warehousing amount within a predetermined range (574).

The purchase and distribution management method for hospital articles can also be embodied as computer readable codes on a computer readable recording medium. The computer readable recording medium is any data storage device that can store data which can be thereafter read by a computer system. Examples of the computer readable recording medium include read-only memory (ROM), random-access memory (RAM), CD-ROMs, magnetic tapes, floppy disks, optical data storage devices, and carrier waves (such as data transmission through the Internet). The computer readable recording medium can also be distributed over network coupled computer systems so that the computer readable code is stored and executed in a distributed fashion.

While the present invention has been particularly shown and described with reference to exemplary embodiments thereof, it will be understood by those of ordinary skill in the art that various changes in form and details may be made therein without departing from the spirit and scope of the present invention as defined by the following claims.

### INDUSTRIAL APPLICABILITY

This invention can be used in the field of managing distribution of the hospital article.

1. A purchase and distribution management system for purchasing articles used in a hospital by proxy and managing the distribution of the articles, the system comprising:
an interface part connecting an exclusive management device of the hospital, the device computerizing the articles in the hospital, to the purchase and distribution management system to be compatible with each other in such a way that a user is able to use the purchase and distribution management system by using only the exclusive management device without downloading a new program;
a purchase application reception part receiving a purchase application for hospital articles from the user;
a contract check part retrieving whether there exists a contract between the user and a provider for the articles;
a contract generation part generating the contract between the user and the provider when there is no contract for the hospital articles for which purchase application is received as a result of the retrieving of the contract check part;
a purchase order management part generating and sending a purchase order for the articles to the provider when there is the contract for the articles for which purchase application is received as the result of the retrieving of the contract check part or the contract is generated by the contract generation part;
a distribution management part checking an amount of each article in stock in the hospital, generating a purchase application according to a result of the checking, and managing a delivery process of the article in the hospital; and
a consignment stock management part allowing a certain amount of warehoused articles for consignment stock to be consigned to the provider to be stored and changing an amount of interim warehoused articles consigned to the provider according to a use of the user.

2. The system of claim 1, further comprising a warehousing check part checking one of warehousing and interim warehousing of articles ordered by the purchase order management part.

3. The system of claim 1, wherein the contract generation part comprises:
a contract type selection part selecting one of a private contract and a tender contract;
a private contract part requesting and receiving an estimate and making a contract with the provider when the contract type selection part selects the private contract;
a tender contract part receiving tenders from a plurality of providers and making a contract when the contract type selection part selects the tender contract; and
a contract approval part receiving an approval of the user with respect to a contract made by one of the private contract part and the tender contract part.

4. The system of claim 3, wherein the tender contract part comprises:
a tender notice generation and providing part generating a notice of tender based on the purchase application of the user and providing the notice of tender to the plurality of providers;
a tender application receiving part receiving tender applications from the plurality of providers; and
a tender examination part examining the tender applications received by the tender application receiving part, accepting one of the tender applications and making a contract with a provider applying the accepted tender application.

5. The system of claim 4, wherein the tender examination part groups a plurality of items, examines the tender applications for each group, accepts one of the tender applications, and makes a contract with the provider applying the accepted tender application.

6. The system of claim 1, wherein the purchase order management part comprises:
a purchase order generation part generating a purchase order on the articles when there is the contract for the articles for which purchase application is received as the result of the retrieving of the contract check part or the contract is generated by the contract generation part;
an electronic payment part allowing the user to excuse an electronic payment with respect to an amount of money written in the purchase order; and
a purchase order sending part sending the purchase order generated by the purchase order generation part to the provider.

7. The system of claim 1, wherein the distribution management part comprises one or more of:
a prescription article management part, when a prescription on a prescription article is inputted, adding the prescription up, generating a delivery list for each department, and executing a daily delivery;
a prepayment article management part, when a prescription on a particular prepayment article is inputted for each patient, generating a purchase application according to the prescription, sending the prescription to the purchase application reception part, and transmitting a prepayment request signal to the purchase order management part;
a quantitative supplement article management part checking a stock reference amount, a use amount, an amount of articles in stock, and a delivery amount for each department with respect to nonprescription articles and executing a delivery of the nonprescription articles to uniformly maintain the stock reference amount of the each department; and
a consumptive article management part receiving an article application with respect to consumptive articles used in hospital from the each department, executing a delivery of a corresponding article, and forming data of the delivery amount.

8. The system of claim 1, wherein the consignment stock management part comprises:
an object article check part checking which article is an object of consignment stock management;
a delivery investigation part investigating a delivery amount of the checked article to the each department;
an interim warehousing generation part sending the purchase application to the purchase application reception part to control a warehousing amount and an interim warehousing amount within a predetermined range, based on information from the delivery investigation part; and
a consignment stock control part checking the ware housing amount and the interim warehousing amount and providing control information to control a ratio between the warehousing amount and the interim warehousing amount within a predetermined range.

9. The system of claim 1, further comprising a standardization code mapping part receiving internal codes of the user and the provider with respect to hospital articles, matching the internal codes with standardization codes, and providing information on the matched standardization codes to the user and the provider.

10. The system of claim 9, wherein the standardization code mapping part comprises:
a code reception part receiving the internal codes of the user and the provider with respect to the hospital articles;
a standardization code database storing standardization codes corresponding to hospital articles;
a code matching part matching the internal code with the standardization code; and
a code relation database storing information on a relation between the matched internal code and standardization code.

11. The system of claim 1, further comprising a barcode management part providing a barcode including information on a hospital article to allow the amount of the hospital articles in stock to be efficiently managed.

12. The system of claim 11, wherein the barcode management part comprises:
a barcode generation part generating the barcode including information on the hospital article; and
a barcode output part outputting and providing the generated barcode.

13. The system of claim 11, wherein the information on the hospital article comprises one or more of a validity period, a contract period, an insurance maximum price, a load position, a price, distribution, and a standardization code of the hospital article.

14. A purchase and distribution management method for purchasing articles used in a hospital by proxy and managing the distribution of the articles by using the system according to claim 1, the method comprising:
connecting an exclusive management device of the hospital, the device computerizing the articles in the hospital, to the purchase and distribution management system to be compatible with each other in such a way that a user is able to use the purchase and distribution management system by using only the exclusive management device without downloading a new program;
receiving a purchase application for hospital articles from the user;
retrieving whether there exists a contract between the user and a provider for the articles;
generating the contract between the user and the provider when there is no contract for the hospital articles for which purchase application is received as a result of the retrieving of the contract check part;
generating and sending a purchase order for the articles to the provider when there is the contract for the articles for which purchase application is received as the result of the retrieving of the contract check part or the contract is generated by the contract generation part;
checking an amount of each article in stock in the hospital, generating a purchase application according to a result of the checking, and managing a delivery process of the article in the hospital; and
allowing a certain amount of warehoused articles for consignment stock to be consigned to the provider to be stored and changing an amount of interim warehoused articles consigned to the provider according to a use of the user.

15. The method of claim 14, further comprising checking one of warehousing and interim warehousing of articles ordered in the generating and sending a purchase order.

16. The method of claim 14, wherein the generating the contract comprises:
selecting one of a private contract and a tender contract;
requesting and receiving an estimate and making a private contract with the provider when the private contract is selected;
receiving tenders from a plurality of providers and making a tender contract when the tender contract is selected; and
receiving an approval of the user with respect to the contract made in the above.

17. The method of claim 16, wherein the receiving tenders from a plurality of providers and making a tender contract comprises:
generating a notice of tender based on the purchase application of the user and providing the notice of tender to the plurality of providers;
receiving tender applications from the plurality of providers; and
examining the received tender applications, accepting one of the tender applications and making a contract with a provider applying the accepted tender application.

18. The method of claim 17, wherein, in the examining the received tender applications, a plurality of items are grouped, the tender applications are examined for each group, one of the tender applications is accepted, and a contract with the provider applying the accepted tender application is made.

19. The method of claim 14, wherein the generating and sending a purchase order comprises:
generating a purchase order on the articles when there is the contract for the articles for which purchase application is received as the result of the retrieving of the contract or the contract is generated in the above;
allowing the user to excuse an electronic payment with respect to an amount of money written in the purchase order; and
sending the generated purchase order to the provider.

20. The method of claim 14, wherein the managing a delivery process comprises one or more of:
when a prescription on a prescription article is inputted, adding the prescription up, generating a delivery list for each department, and executing a daily delivery;
when a prescription on a particular prepayment article is inputted for each patient, generating a purchase application according to the prescription, sending the prescription to the purchase application reception part, and transmitting a prepayment request signal to the purchase order management part;
checking a stock reference amount, a use amount, an amount of articles in stock, and a delivery amount for each department with respect to nonprescription articles and executing a delivery of the nonprescription articles to uniformly maintain the stock reference amount of the each department; and
receiving an article application with respect to consumptive articles used in hospital from the each department, executing a delivery of a corresponding article, and forming data of the delivery amount.

21. The method of claim 14, wherein the allowing a certain amount of warehoused articles for consignment stock to be consigned to the provider comprises:
checking which article is an object of consignment stock management;
investigating a delivery amount of the checked article to the each department;
sending the purchase application to the purchase application reception part to control a warehousing amount and an interim warehousing amount within a predetermined range, based on information from the investigating a delivery amount; and
checking the ware housing amount and the interim warehousing amount and providing control information to control a ratio between the warehousing amount and the interim warehousing amount within a predetermined range.

22. The method of claim 14, further comprising receiving internal codes of the user and the provider with respect to hospital articles, matching the internal codes with standardization codes, and providing information on the matched standardization codes to the user and the provider.

23. The method of claim 22, wherein the receiving internal codes of the user and the provider with respect to hospital articles, matching the internal codes with standardization codes, and providing information on the matched standardization codes to the user and the provider comprises:
receiving the internal codes of the user and the provider with respect to the hospital articles;
storing standardization codes corresponding to hospital articles in a standardization code database;
matching the internal code with the standardization code; and
storing information on a relation between the matched internal code and standardization code in a code relation database.

24. The method of claim 14, further comprising providing a barcode including information on a hospital article to allow the amount of the hospital articles in stock to be efficiently managed.

25. The method of claim 24, wherein the providing a barcode including information on a hospital article to allow the amount of the hospital articles in stock to be efficiently managed comprises:
generating the barcode including information on the hospital article; and
outputting and providing the generated barcode.

26. The method of claim 24, wherein the information on the hospital article comprises one or more of a validity period, a contract period, an insurance maximum price, a load position, a price, distribution, and a standardization code of the hospital article.
